(19) United States
(12) Patent Application Publication
(10) Pub. No.: US 2020/0093131 A1
(43) Pub. Date: Mar. 26, 2020

(54) STABILISED FORMULATIONS CONTAINING PROTHIOCONAZOLE WITH A LOW 2-(1-CHLOROCYCLOPROPYL)-1-(2-CHLOROPHENYL)-3-(1H-1,2,4-TRIAZOL-1-YL)PROPAN-2-OL CONTENT

(71) Applicant: BAYER AKTIENGESELLSCHAFT, Leverkusen (DE)

(72) Inventors: Jens KRAUSE, Leverkusen (DE); Martin WEISS, Bornheim/Sechtem (DE); Martin STEINBECK, Koeln (DE)

(21) Appl. No.: 16/620,027

(22) PCT Filed: Jun. 6, 2018
(86) PCT No.: PCT/EP2018/064894

§ 371 Date: Dec. 6, 2019

(30) Foreign Application Priority Data
Jun. 12, 2017 (EP) 17175390.8

(51) Int. Cl. A01N 43/653; A01N 25/04; A01N 43/56; A01N 25/22; C07C 233/38; C07C 317/28

(52) U.S. Cl. CPC A01N 43/653; A01N 25/04; C07C 317/28; A01N 25/22; C07C 233/38; A01N 43/56

(57) ABSTRACT

The invention relates to storage-stable prothioconazole-containing formulations having a particularly low content of 2-(1-chlorocyclopropyl)-1-(2-chlorophenyl)-3-( H-1,2,4-triazol-1-yl)propan-2-ol based on organic vinyl compounds, to a process for their preparation, to a method for controlling phytopathogenic fungi in crop protection and to their use as crop protection agents.

The invention relates to storage-stable prothioconazole-containing formulations having a particularly low content of 2-(1-chlorocyclopropyl)-1-(2-chlorophenyl)-3-(1H-1,2,4-triazol-1-yl)propan-2-ol based on organic vinyl compounds, to a process for their preparation, to a method for controlling phytopathogenic fungi in crop protection and to their use as crop protection agents.

It is already known that prothioconazole can be used in standard formulations for control of fungi (WO-A 96/16 048). This active ingredient is 2-[2-(1-chlorocyclopropyl)-3-(2-chlorophenyl)-2-hydroxypropyl]-2,4-dihydro-3H-1,2,4-triazole-3-thione. Prothioconazole-containing formulations are generally liquid formulations and are supplied on the market, for example, in the form of emulsion concentrates.

It is known that the active ingredient prothioconazole can be degraded under particular conditions to give the compound 2-(1-chlorocyclopropyl)-1-(2-chlorophenyl)-3-(1H-1,2,4-triazol-1-yl)propan-2-ol (PSM-Zulassungsbericht [Crop Protection Agent Registration Report], Tilmor, 2010.08.30, serial no. 21, German Federal Office of Consumer Protection and Food Safety).

Therefore, prothioconazole (hereinbelow referred to as PTZ)-containing formulations, as early as in the course of production, may contain a certain amount of 2-(1-chlorocyclopropyl)-1-(2-chlorophenyl)-3-(1H-1,2,4-triazol-1-yl)propan-2-ol. In the case of storage under severe conditions, such as elevated temperatures, incidence of light and intensive oxygen contact, it is likewise possible for degradation of prothioconazole to take place to give 2-(1-chlorocyclopropyl)-1-(2-chlorophenyl)-3 -(1H-1,2,4-triazol-1-yl)propan-2-ol, as a result of which the proportion of active ingredient in the formulations is correspondingly reduced. Since the compound 2-(1-chlorocyclopropyl)-1-(2-chlorophenyl)-3-(1H-1,2,4-triazol-1-yl)ppropan-2-ol (hereinbelow referred to as desthio) is a relevant impurity, the content thereof in prothioconazole-containing formulations is subject to regulatory limitation.

Here, the maximum permissible amount of desthio in PTZ formulations depends on the amount of PTZ in the formulation in g/l. The maximum permissible amount of desthio is given in ppm, with the permissible proportion of desthio in ppm being half of the value of the PTZ load in g/l. Thus, the maximum permissible desthio content in a formulation with 100 g/l PTZ is 50 ppm (=0.005% by weight).

WO-A 2012/033590 discloses aqueous dispersions of prothioconazole containing a sulfur compound, for example L-cysteine, for stabilization.

The unpublished PCT/EP 2016/080215 (Bayer CropScience AG) is considered to be the closest prior art. What is described therein are emulsion concentrates comprising PTZ dissolved in a solvent. It was possible to stabilize the formulation by addition of formula (I)

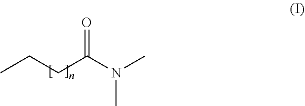

in which
n represents 3, 4, 5, 6, 7, 8, 9, 10, 11, 12, 15 or 18
and to achieve a reduction of the desthio content.

Compounds of the formula (I) are known from WO-A 2012/061094 and can be prepared by the process of metathesis. These are, for example, products from Stepan ((N,N-dimethyl9-decenamide, CAS number: 1356964-77-6, Hallcomid® 1025 or Steposol® MET-10U).

These compounds have the disadvantage that they can only be prepared via a technically complex olefin metathesis step, that they comprise unwanted metal impurities which may catalyze the decomposition of active and inactive ingredients, and that compound I can only be used as solvent. Thus, alternative formulation types where PTZ is not dissolved (such as, for example, WG, SC, OD) are not accessible. Another disadvantage is the limited availability of products. Hitherto, the only industrial quantities of compound (I) available are of Hallcomid 1025.

WO 2016/092030 describes sugar surfactants having a 9-decenoyl radical for use in aqueous adjuvant compositions for increasing the efficacy of active electrolyte ingredients. Hereinbelow, the compounds described in WO 2016/092030 are referred to as glycoside surfactants having a 9-decenoyl radical.

There was therefore a need for stable PTZ-containing formulations, which, even over a relatively long period and under unfavorable storage conditions, for example oxygen contact, high temperatures or incidence of light, do not include any amounts of PTZ degradation products, in particular of 2-(1-chlorocyclopropyl)-1-(2-chlorophenyl)-3-(1H-1,2,4-triazol-1-yl1)propan-2-ol.

Accordingly, it was an object of the present invention to provide novel, improved prothioconazole-containing formulations which have high storage stability and do not exhibit any significant degradation rates of prothioconazole to give 2-(1-chlorocyclopropyl)-1-(2-chlorophenyl)-3-(1H-1,2,4-triazol-1-yl)propan-2-ol, where the formulations are free of compounds of formula (I).

In addition, the formulations are preferably free of glycoside surfactants having a 9-decenoyl radical. The present invention therefore provides formulations comprising
a) prothioconazole and
b) at least one compound of the formula (IV)

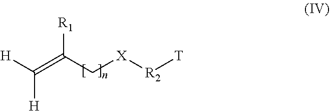

where

n=0-24; preferably 2-16; particularly preferably 4-14; and very particularly preferably 8-12:

R₁═H, C₁-C₆-alkyl, —CN, Cl, Br, F; preferably H, C₁-C₄-alkyl, more preferably H, methyl; and particularly preferably H;

X═—COO—, —CONR₃R₄, —S—, —SO—, —O— and —COS—; preferably —COO—, CONR₃R₄, —S—, —O—and —COS—; more preferably —COO—, CONR₃R₄; and particularly preferably —COO—;

R_{2═}(—CH₂O—)ₘ, (—CH₂O—)ₘ, (—C₂H₄O—)ₘ, (—C₃H₆)ₘ, (—C₄H₈O—)ₘ; preferably (—CH₂—)ₘ, (—CH₂O—)ₘ, (—C₂H₄O—)ₘ, (—C₃H₆O—)ₘ; more preferably (—CH₂O—)ₘ, (—C₂H₄)ₘ, (—C₃H₆O—)ₘ; and particularly preferably (—CH₂O—)ₘ,

T=H, sulfate, phosphate, C₁-C₃-alkyl, —OH, —SH, sulfone; preferably H, sulfate, sulfone, —OH, phosphate;

more preferably H, sulfate, —OH; and particularly preferably H and OH;

R₃═H, C₁-C₆-alkyl, C₃-C₆cycloalkyl, C₆-C₁₀-aryl, preferably H, C₁-C₄-alkyl, C₅-C₆-cycloalkyl, phenyl, more preferably H, methyl and phenyl; and particularly preferably H;

R₄═H, methyl, ethyl, propyl, (—CH₂—)ₘ, (—CH₂O—)ₘ, (—C₂H₄O—)ₘ, (—C₃H₆O—)ₘ, (—C₄H₈O—)ₘ; preferably H, methyl, ethyl, propyl, —CH₂—)ₘ, (—CH₂O—)ₘ, (—C₂H₄O—)ₘ, (—C₃H₆O—); more preferably H, methyl, ethyl, propyl, (—CH₂O—)ₘ, (—C₂H₄O—)ₘ, (—C₃H₆O—)ₘ; and particularly preferably H, methyl, ethyl, propyl, (—CH₂O—)ₘ,

and R₃ is different from R₄, and

m=0-100, preferably 2-50, more preferably 5-25, and particularly preferably 8-12;

where the formulation is free from compounds according to formula (I)

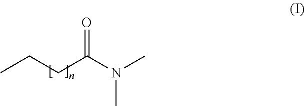

in which
n represents 3, 4, 5, 6, 7, 8, 9, 10, 11, 12, 15 or 18,

and additionally free from glycoside surfactants having a 9-decenoyl radical,

where

for X=—CNR₃R₄ and m=0 T is not present.

The abovementioned radical definitions, for example C₃H₆O, also include the structural isomers comprised by this empirical formula, which will be evident to the person skilled in the art. Furthermore, in terms such as “sulfate, phosphate, etc.” the respective radicals are to be understood as radicals where the corresponding acids may also be present as salts. Preferred as counterions for this purpose are alkali metal ions, calcium, isopropylammonium and ammonium; however, the use of other counterions known to the person skilled in the art is also possible.

In a preferred embodiment, the present invention provides a formulation comprising
a) prothioconazole and
b) at least one compound of the formula (IV)

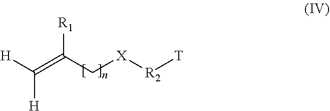

where

n=2-16;

R₁=H, C₁-C₄-alkyl,

X═—COO—, CONR₃R₄, —S—, —O— and —COS—; R₂═(—CH₂—)ₘ, (—CH₂O—)ₘ, (—C₂H₄O—)ₘ, (—C₃H₆O—)ₘ;

T=H, sulfate, sulfone, —OH, phosphate;

R₃═H, C₁-C₄-alkyl, C₅-C₆-cycloalkyl, phenyl;

R₄═H, methyl, ethyl, propyl, (—CH₂—)ₘ, (—CH₂O—)ₘ, (—C₂H₄O—)ₘ, (—C₃H₆O—)ₘ;

and R₃ is different from R₄, and

m=0-50;

where the formulation is free from compounds according to formula (I)

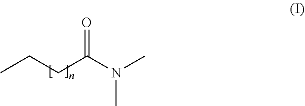

in which
n represents 3, 4, 5, 6, 7, 8, 9, 10, 11, 12, 15 or 18,

and additionally free from glycoside surfactants having a 9-decenoyl radical,

where

for X═—CNR₃R₄ and m=0 T is not present.

In a further preferred embodiment, the present invention provides a formulation comprising
a) prothioconazole and
b) at least one compound of the formula (IV)

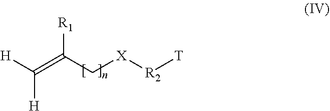

where

n=4-14;

R₁═H, methyl; X=—COO—, CONR₃R₄;

R₂═(—CH₂O—)ₘ, (—C₂H₄O—)ₘ, (—C₃H₆O—)ₘ;

T=H, sulfate, —OH;

R₃═H, methyl and phenyl;

R₄═H, methyl, ethyl, propyl, (—CH₂O—)ₘ, (—C₂H₄O—)ₘ, (—C₃H₆O—)ₘ;

and R₃ is different from R₄, and

m=0-25,

where the formulation is free from compounds according to formula (I)

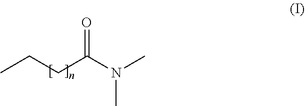

in which
n represents 3, 4, 5, 6, 7, 8, 9, 10, 11, 12, 15 or 18,

and additionally free from glycoside surfactants having a 9-decenoyl radical,

where

for X=—CNR₃R₄ and m=0 T is not present.

In a particularly preferred embodiment, the present invention provides a formulation comprising
a) prothioconazole and
b) at least one compound of the formula (IV)

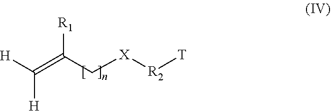

where

n=8-12:

R₁═H;

X═—COO—,

R_{2═}(—CH₂O—)ₘ,

T=H and OH;

m=8-12;

where the formulation is free from compounds according to formula (I)

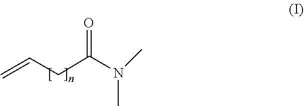

in which
n represents 3, 4, 5, 6, 7, 8, 9, 10, 11, 12, 15 or 18,

and additionally free from glycoside surfactants having a 9-decenoyl radical.

In an alternative preferred embodiment where n=7 and m=0, the present invention provides a formulation comprising
a) prothioconazole and
b) at least one compound of the formula (IV)

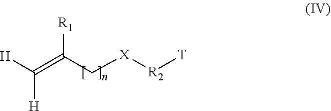

where

n=7:

R₁═H;

X=—COO—;

T=H;

m=0;

where the formulation is free from compounds according to formula (I)

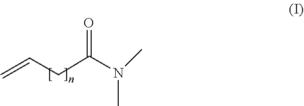

in which
n represents 3, 4, 5, 6, 7, 8, 9, 10, 11, 12, 15 or 18,

and additionally free from glycoside surfactants having a 9-decenoyl radical.

In the embodiments mentioned above, R₂ may also be a copolymer, preferably a block copolymer of at least two monomers selected from (—CH₂—)ₘ, (—CH₂O—)ₘ, (—C₂H₄O—)ₘ, (—C₃H₆O—)ₘ, (—C₄H₈O—)ₘ, in this case, R₂ is preferably a block copolymer selected from (—C₂H₄O—)ₘ, (—C₃H₆O—)ₘ.

In a preferred embodiment, the compound IV is selected from the group comprising

9-decenoic acid, 9-decenoate ester having n EO groups (EO═C₂H₄O), where n=8, 9, 10, 11 and preferably n=10;

C10-17 DMAPA-amides

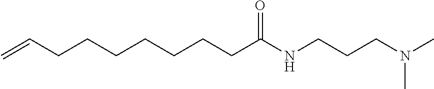 [SMILES: C=CCCCCCCCC(=O)NCCCN(C)C]

C10-20 DMAPA amine-oxides

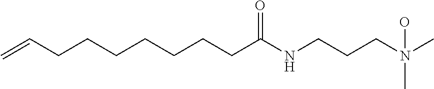

and

C10-24: sulfobetaines.

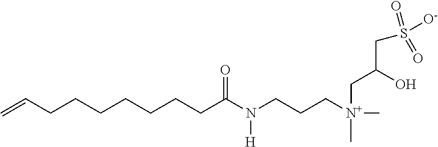 [SMILES: C=CCCCCCCCC(=O)NCCC[N+](C)(C)CC(O)CS(=O)(=O)[O-]]

Prothioconazole (with the chemical name 2-[2-(1-chlorocyclopropyl)-3-(2-chlorophenyl)-2-hydroxypropyl]1-1,2-dihydro-3H-1,2,4-triazole-3-thione) (CAS number 178928-70-6) takes the form of a racemate. Suitable processes for preparation thereof are described in DE-A 195280. Prothioconazole may be present in the thiono form of the general formula (II)

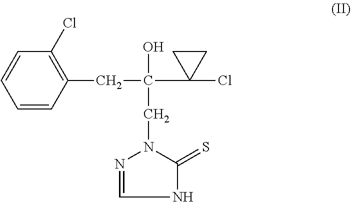

or in the tautomeric mercapto form of the general formula (IIa).

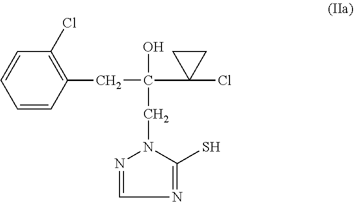

The use of the term “prothioconazole” hereinafter always covers the isomers shown and further possible tautomers.

2-(1-Chlorocyclopropyl)-1-(2-chlorophenyl)-3-(1H-1,2,4-triazol-1-yl)propan-2-ol is also present as a racemate and has the general formula (III), where in turn all tautomeric forms are to be embraced by this representation.

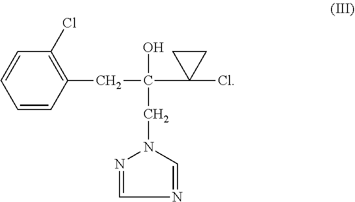

The proportion of component a) (prothioconazole) in the formulations according to the invention is preferably 1% by weight to 50% by weight, more preferably 4% by weight to 30% by weight, and particularly preferably 5% by weight to 28% by weight.

The proportion of component b) in the formulations according to the invention is preferably 1% by weight to 50% by weight, more preferably 2% by weight to 45% by weight, even more preferably 5% by weight to 35% by weight and particularly preferably 8% by weight to 25% by weight.

Furthermore, the formulations according to the present invention, as described above, may each comprise further ingredients, such as:
c) c1: non-ionic dispersant/emulsifier,
c2: ionic dispersant/emulsifier
d) other active agrochemical ingredients different from a)
e) solvents including OD carriers
f) carriers (WGs, and also Aerosils for SC/TC/WG)
g) g1: organic thickeners
g2: inorganic thickeners
h) further additives and auxiliaries

Formulation types are defined by the FAO and are found at www.fao.org/ag/agp/aspp/pesticid. In the March 2016 edition, the customary formulation types are described on pages 66-231. The formulation according to the invention is a customary formulation type described by the FAO. Examples which may be mentioned here are suspension concentrates (SC), and also colorant-containing concentrates for seed treatment (FS), emulsion concentrates (EC), water-dispersible concentrates (WG), oil dispersions (OD), suspoemulsions (SE), aqueous emulsions (EW), microemulsions (ME) and liquid formulations (SL). Preferred are EC, SC, FS, SE, OD and WG formulation types, very particular preference is given to formulations where at least one active ingredient is not dissolved. Very particular preference is given to FS, SC, SE, OD and WG formulations, most preferably SC, FS and WG formulations.

The present invention further provides for the use of the formulations of the invention for treatment of plants and corresponding methods.

In a preferred embodiment, IV is not a solvent for component a).

Nonionic Emulsifiers and Dispersants c1)

Useful nonionic emulsifiers and dispersants c1), such as emulsifiers, wetting agents, surfactants and dispersers, include standard surface-active substances present in formulations of active agrochemical ingredients. Examples include ethoxylated nonylphenols, reaction products of linear or branched alcohols with ethylene oxide and/or propylene oxide, ethylene oxide-propylene oxide block copolymers, end group-capped and non-end group-capped alkoxylated linear and branched, saturated and unsaturated alcohols (e.g. butoxy polyethylenepropylene glycols), reaction products of alkylphenols with ethylene oxide and/or propylene oxide, ethylene oxide-propylene oxide block copolymers, polyethylene glycols and polypropylene glycols, and also fatty acid esters, fatty acid polyglycol ether esters, alkylsulfonates, alkylsulfates, arylsulfates, ethoxylated arylalkylphenols, for example tristyrylphenol ethoxylate having an average of 16 ethylene oxide units per molecule, and also ethoxylated and propoxylated arylalkylphenols, and also sulfated and phosphated arylalkylphenol ethoxylates or ethoxy- and propoxylates. Particular preference is given to tristyrylphenol alkoxylates and fatty acid polyglycol ether esters. Very particular preference is given to tristyrylphenol ethoxylates, tristyrylphenol ethoxy propoxylates and castor oil polyglycol ether esters, in each case individually or in mixtures. Additives may additionally be useful, such as surfactants or esters of fatty acids, which contribute to improvement in biological efficacy. Suitable nonionic emulsifiers and dispersants cl) are, for example, Soprophor® 796/P, Lucramul® CO30, Lucramul® HOT, Lucramul® PSI 100 or Synperonic® T304.

Suitable nonionic dispersers c1) may likewise be selected from the group comprising polyvinylpyrrolidone (PVP), polyvinyl alcohol, copolymer of PVP and dimethylaminoethyl methacrylate, butylated PVP, copolymer of vinyl chloride and vinyl acetate, and partially hydrolyzed vinyl acetate, phenol resins, modified cellulose types, for example Luviskol® (polyvinylpyrrolidone), Mowiol® (polyvinyl alcohol) or modified cellulose. Preference is given to polyvinylpyrrolidone types, particular preference to types of low molecular weight such as Luviskol® K30 or Sokalan® K30.

Useful further nonionic emulsifiers and dispersants c1) from the group of the di- and triblock copolymers of alkylene oxides are, for example, compounds based on ethylene oxide and propylene oxide, having mean molar masses between 200 and 10 000 and preferably 1000 to 4000 g/mol, where the proportion by mass of the polyethoxylated block varies between 10 and 80%, for example the Synperonic® PE series (Uniqema), the Pluronic® PE series (BASF), the VOP® 32 or Genapol® PF series (Clariant).

The proportion of nonionic emulsifiers and dispersants c1) required in the suspension concentrates according to the invention is preferably 1% to 15% by weight, more preferably 2% to 10% by weight and particularly preferably 2.5% to 8% by weight.

Anionic Emulsifiers and Dispersants c2)

Suitable anionic emulsifiers and dispersants b1), such as emulsifiers, surfactants, wetting agents and dispersers, are, for example, alkali metal, alkaline earth metal or ammonium salts of sulfonates, sulfates, phosphates, carboxylates and mixtures thereof, for example the salts of alkylsulfonic acids or alkylphosphoric acids and alkylarylsulfonic or alkylarylphosphoric acids, diphenylsulfonates, alpha-olefinsulfonates, lignosulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols or of fatty acid esters. Examples of phosphates are phosphate esters. Examples of carboxylates are alkyl carboxylates and carboxylated alcohol ethoxylates or alkylphenol ethoxylates. Likewise suitable is the group of anionic emulsifiers of the alkali metal, alkaline earth metal and ammonium salts of the polystyrenesulfonic acids, salts of the polyvinylsulfonic acids, salts of the alkylnaphthalenesulfonic acids, salts of alkylnaphthalenesulfonic acid-formaldehyde condensation products, salts of condensation products of naphthalenesulfonic acid, phenolsulfonic acid and formaldehyde. Examples are calcium dodecylbenzenesulfonate such as Rhodocal® 70/B (Solvay), Phenylsulfonat CA100 (Clariant) or isopropylammonium dodecylbenzenesulfonates such as Atlox® 3300B (Croda).

Further typical representatives include Phenylsulfonat CA (calcium dodecylbenzenesulfonate), Soprophor® products (optionally esterified derivatives of tristyrylphenol ethoxylates), Emulsogen® 3510 (alkylated EO/PO copolymer), Emulsogen® EL 400 (ethoxylated castor oil), Tween® products (fatty acylated sorbitan ethoxylates), Calsogen AR 100 (calcium dodecylbenzenesulfonate). Preference is given to combinations of salts of alkylated aromatic sulfonic acids, such as calcium phenylsulfonate and/or Calsogen® AR 100, with alkylated copolymers of ethylene oxide and propylene oxide, such as Emulsogen® 3510. Particular preference is given to combinations of salts of dodecylbenzenesulfonic acid, such as Calsogen® AR 100, with alkylated copolymer of ethylene oxide and propylene oxide, such as Emulsogen® 3510.

Examples of further anionic emulsifiers and dispersants c2) from the group of the naphthalenesulfonates are Galoryl® MT 800 (sodium dibutylnaphthalenesulfonate), Morwet® IP (sodium diisopropylnaphthalenesulfonate) and Nekal® BX (alkylnaphthalenesulfonate). Examples of anionic surfactants from the group of the condensates of naphthalenesulfonates with formaldehyde are Galoryl® DT 201 (naphthalenesulfonic acid hydroxy polymer with formaldehyde and methylphenol sodium salt), Galoryl® DT 250 (condensate of phenol- and naphthalenesulfonates), Reserve® C (condensate of phenol- and naphthalenesulfonates) or Morwet® D-425, Tersperse® 2020. Preference is given to 1,2-dibutyl- or -diisobutyl-substituted naphthalenesulfonates, for example products such as Galoryl® MT 800 (CFPI-Nufarm) and Nekal® BX (BASF). Further typical surfactants are Soprophor® 3D33, Soprophor® 4D384, Soprophor® BSU, Soprophor® CY/8 (Solvay) and Hoe® S3474, and in the form of the Sapogenat® T products (Clariant), for example Sapogenat® T 100.

The proportion of anionic emulsifiers and dispersants c2) required in the technical concentrates according to the invention is preferably 2% to 35% by weight, more preferably 3% to 30% by weight, even more preferably 5% to 25% by weight and particularly preferably 10% to 20% by weight.

The proportion of anionic emulsifiers and dispersants c2) required in the suspension concentrates according to the invention is preferably 0.1% to 10% by weight, more preferably 0.2% to 7% by weight and particularly preferably 0.3% to 4% by weight.

Further Active Agrochemical Ingredients Different from PTZ d):

Further active agrochemical ingredients d) in the context of the present invention are active fungicidal, insecticidal or herbicidal ingredients. In an alternative embodiment, the formulation of the invention comprises one or more further active insecticidal or fungicidal ingredients d), more preferably one or more active fungicidal ingredients d). The active ingredients used are preferably water-insoluble.

The Pesticide Manual provides a review of typical crop protection agents.

Preferred insecticidal components d) are, for example, imidacloprid, nitenpyram, acetamiprid, thiacloprid, thiamethoxam, clothianidin, cyantraniliprole, chlorantraniliprole, flubendiamide, tetraniliprole, cyclaniliprole, spirodiclofen, spiromesifen, spirotetramat, abamectin, acrinathrin, chlorfenapyr, emamectin, ethiprole, fipronil, flonicamid, flupyradifurone, indoxacarb, metaflumizone, methoxyfenozid, milbemycin, pyridaben, pyridalyl, silafluofen, spinosad, sulfoxaflor, triflumuron, the compound from WO-A 2006/089633 as Example I-1-a-4, the compound disclosed in WO-A 2008/067911 as Example I-1-a-4, the compound disclosed in WO 2013/092350 as Example Ib-14, the compound disclosed in WO 2010/51926 as Example Ik-84.

Preferred fungicidal components d) are, for example, bixafen, fenamidone, fenhexamid, fluopicolide, fluopyram, fluoxastrobin, iprovalicarb, isotianil, isopyrazam, pencycuron, penflufen, propineb, tebuconazole, trifloxystrobin, ametoctradin, amisulbrom, azoxystrobin, benthiavalicarb-isopropyl, benzovindiflupyr, boscalid, carbendazim, chlorothanonil, cyazofamid, cyflufenamid, cymoxanil, cyproconazole, difenoconazole, ethaboxam, epoxiconazole, famoxadone, fluazinam, fluquinconazole, flusilazole, flutianil, fluxapyroxad, isopyrazam, kresoxim-methyl, lyserphenvalpyr, mancozeb, mandipropamid, metconazol, pyriofenone, folpet, metaminostrobin, oxathiapiprolin, penthiopyrad, picoxystrobin, probenazole, proquinazid, pydiflumetofen, pyraclostrobin, sedaxane, spiroxamin, tebufloquin, tetraconazole, valiphenalate, zoxamide, ziram, N-(5-chloro-2-isopropylbenzyl)-N-cyclopropyl-3-(difluoromethyl)-5-fluoro-1-methyl-1H-pyrazole-4-carboxamide, N-(5-chloro-2-isopropylbenzyl)-N-cyclopropyl-3-(difluoromethyl)-5-fluoro-1-methyl-1H-pyrazole-4-carboxamide, 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, 2-{3-[2-1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenylmethane-sulfonate, (3S,6S,7R,8R)-8-benzyl-3-[({3-[(isobutyryloxy)methoxy]-4-methoxypyridin-2-yl}carbonyl)-amino]-6-methyl-4,9-dioxo-1,5-dioxonan-7-yl 2-methylpropanoate (lyserphenvalpyr).

Particularly preferred fungicidal mixing partners d) for prothioconazole are, for example: tebuconazole, spiroxamin, bixafen, fluoxastrobin, trifloxystrobin, N-(5-chloro-2-isopropylbenzyl)-N-cyclopropyl-3-(difluoromethyl)-5-fluoro-1-methyl-1H-pyrazole-4-carboxamide, (3S,6S,7R,8R)-8-benzyl-3[({3-[(isobutyryloxy)methoxy]-4-methoxypyridin-2-yl}carbonyl)amino]-6-methyl-4,9-dioxo-1,5 -dioxonan-7-yl 2-methylpropanoate (lyserphenvalpyr) and fluopyram.

Very particular preference is given to the mixtures of a) (prothioconazole) with one or more compounds selected from the group of the compounds d):
a) +d) tebuconazole;
a) +d) trifloxystrobin;
a) +d) fluoxastrobin;
a) +d) bixafen;
a) +d) fluopyram
a) +d) spiroxamine;
a) +d) fluoxastrobin+trifloxystrobin;
a) +d) trifloxystrobin+spiroxamine;
a) +d) bixafen+tebuconazole;
a) +d) bixafen+fluoxastrobin;
a) +d) bixafen+trifloxystrobin;
a) +d) bixafen+fluopyram;
a) +d) tebuconazole+spiroxamine;
a) +d) tebuconazole+fluopyram;
a) +d) N-(5-chloro-2-isopropylbenzyl)-N-cyclopropyl-3-(difluoromethyl)-5-fluoro-1-methyl-1H-pyrazole-4-carboxamide
a) +d) N-(5-chloro-2-isopropylbenzyl)-N-cyclopropyl-3-(difluoromethyl)-5-fluoro-1-methyl-1H-pyrazole-4-carboxamide+tebuconazole
a) +d) N-(5-chloro-2-isopropylbenzyl)-N-cyclopropyl-3-(difluoromethyl)-5-fluoro-1-methyl-1H-pyrazole-4-carboxamide+flupyram
a) +d) bixafen+(3S,6S,7R,8R)-8-benzyl-3-[({3-[isobutyryloxy)methoxy]-4-methoxypyridin-2-yl}carbonyl)amino]-6-methyl-4,9-dioxo-1,5- dioxonan-7-yl 2-methylpropanoate (lyserphenvalpyr)

The proportion of component d) in the formulations of the invention is preferably 1% by weight to 40% by weight, particularly preferably 3% by weight to 35% by weight.

Solvents e):

The formulations may comprise solvents e), for example in the case of EC, OD or SE formulations.
aromatic hydrocarbon mixtures (preferably naphaline reduced), for example Solvesso®;
aromatic hydrocarbons, for example white spirit, petroleum, alkylbenzenes and spindle oil, xylene, toluene or alkylnaphthalenes;
aliphatic/cycloaliphatic hydrocarbons, for example mineral oils, hexane, heptane, octane, nonane, decane, cyclopentane, cyclohexane, decalin or white oil;
chlorinated aromatic or aliphatic hydrocarbons, for example chlorobenzene, chloroethylene, or methylene chloride, chloroform or tetrachloromethanes;
alcohols, for example methanol, ethanol, isopropanol, butanol, ethylene glycol, propylene glycol or benzyl alcohol;
ethers, for example tetrahydrofuran, tetrahydrofurfuryl alcohol, tetrahydropyran, 1,4-dioxanes, diethyl ether, methyl tert-butyl ether, dihexyl ether, dioctyl ether, didecyl ether, dibenzyl ether, dimethylisosorbide, diphenyl ether, ethyl phenyl ether, phenyl benzyl ether or anisole;
monoesters/diesters/glycerol esters, for example ethyl acetate, butyl propionate, pentyl propionate, benzyl acetate, benzyl benzoate, butyl benzoate, Rhodiasolv® Polarclean, Rhodiasolv® RPDE, methyl lactate, ethyl lactate, propyl lactate, butyl lactate, 2-ethylhexyl lactate, ethyl 3-ethoxypropionate (UCAR® Ester EEP, Dow Chemical Company), dimethyl succinate, diethyl succinate, dipropyl succinate, dimethyl adipate, diethyl adipate, dipropyl adipate, dimethyl glutarate, diethyl glutarate, dipropyl glutarate, bis(2-ethylhexyl) adipate, diisopropyl adipate, dimethyl 2-methylglutarate, dioctyl maleate, glycerol monoacetate, glycerol diacetate, glycerol triacetate, vegetable oils, for example rapeseed oil, sunflower oil, soya oil, castor oil or corn oil;
lactones, for example butyrolactone, alpha-methyl-gamma-butyrolactone, gamma-valerolactone or delta-valerolactone;
polyethylene/propylene oxides, for example monoethylene glycol, monoethylene glycol monomethyl ether, monoethylene glycol monoethyl ether, monoethylene glycol monopropyl ether, monoethylene glycol monobutyl ether, monoethylene glycol monopentyl ether, monoethylene glycol monohexyl ether, monoethylene glycol monophenyl ether, monoethylene glycol dimethyl ether, monoethylene glycol diethyl ether, monoethylene glycol dipropyl ether, monoethylene glycol dibutyl ether, monoethylene glycol dipentyl ether, monoethylene glycol dihexyl ether, monoethylene glycol diphenyl ether, and the longer ethylene glycol homologs thereof; monopropylene glycol, monopropylene glycol monomethyl ether, monopropylene glycol monoethyl ether, monopropylene glycol monopropyl ether, monopropylene glycol monobutyl ether, monopropylene glycol monopentyl ether, monopropylene glycol monohexyl ether, monopropylene glycol monophenyl ether, monopropylene glycol dimethyl ether, monopropylene glycol diethyl ether, monopropylene glycol dipropyl ether, monopropylene glycol dibutyl ether, monopropylene glycol dipentyl ether, monopropylene glycol dihexyl ether, monopropylene glycol diphenyl ether, and the longer propylene homologs thereof; monoethylene glycol monomethyl ether acetate, monoethylene glycol diacetate, and the longer ethylene glycol homologs thereof; monopropylene glycol monomethyl ether acetate, monopropylene glycol acetate, and the longer polypropylene glycol homologs thereof;
simple and substituted amines, for example diethylamines, triethylamines, diisopropylamines, diisopropylethylamines, monoethanolamines, diethanolamines, triethanolamines and more highly alkoxylated amines, anilines or dimethylanilines;
amides/ureas, for example N-formylmorpholine, N,N-dimethylformamide, N,N-dimethylacetamide, N,N-dimethylbenzamide, N,N-dimethyloctanamide, N,N-dimethyldecanamide, N,N-dimethyldec-9-en-l-amide, N,N-dimethyldodedecanamide, N,N-dimethyllactamide, N,N-decylmethylformamide, N-methyl-2-pyrrolidone, N-ethyl-2-pyrrolidone,

N-propyl-2-pyrrolidone, N-butyl-2-pyrrolidone, N-pentyl-2-pyrrolidone, N-hexyl-2-pyrrolidone, N-heptyl-2-pyrrolidone, N-octyl-2-pyrrolidone, N-nonyl-2-pyrrolidone, N-decyl-2-pyrrolidone, N-undecenyl-2-pyrrolidone, N-dodecyl-2-pyrrolidone, N-methyl-2-piperidone, N-methylcaprolactam, N-octylcaprolactam, tetramethylurea, tetraethylurea, 1,3-dimethyl-2-imidazolidinone, 1,3,4-trimethyl-2-imidazolidinone, 1,3-dimethyl-3,4,5,6-tetrahydro-2(1H)-pyrimidinone 1-heptyl-3-methyl-2-imidazolidinone, 1-heptyl-1,3-dihydro-3-methyl-2H-imidazol-2-one;

ketones, for example acetone, diacetone alcohol, methyl ethyl ketone, 2-pentanone, 3-pentanone, 2-hexanone, 3-hexanone, 2-heptanone, 3-heptanone, 4-heptanone, 2-octanone, 3-octanone, 4-octanone, methyl isopropyl ketone, methyl isobutyl ketone, methyl isopentyl ketone, ethyl isopropyl ketone, ethyl isobutyl ketone, ethyl isopentyl ketone, propyl isopropyl ketone, propyl isobutyl ketone, propyl isopentyl ketone, 3,3-dimethyl-2-butanone, 2,4-dimethyl-3-pentanone, 4,4-dimethyl-2-pentanone, 2,6-dimethyl-4-heptanone, 2,2,4,4-tetramethyl-3-pentanone, cyclopentanone, cyclohexanone. cycloheptanone, cyclooctanone, 2,4,6-cycloheptatrien-1-one, acetophenone, propiophenone, 1-(4-methylphenyl)ethanone, 1-(4-ethylphenyl)ethanone, 2-methyl-1-phenyl-1-propanone, 1-(3-ethylphenyl)ethanone, 4-phenyl-2-butanone, 1-phenyl-2-propanone, 1-phenyl-2-butanone, 2-phenyl-3-butanone, butyrophenone or valerophenone.
nitriles, for example acetonitrile, propanenitrile, 2-methylpropanenitrile, butanenitrile, 3-methylbutanenitrile, pentanenitrile, 4-methylpentanenitrile, hexanenitrile, heptanenitrile, octanenitrile, nonanenitrile, decanenitrile, benzonnitrile, benzeneacetonitrile, pentanedinitrile, 2-methylpentanedinitrile, hexanedinitrile, heptanedinitrile, octanedinitrile or nonanedinitrile;

acetals, for example 1,1-dimethoxymethane; 1,1-dimethoxyethane; 1,1′-[methylenebis(oxy)]bisethane; 1,1-diethoxyethane; 1,1′-[methylenebis(oxy)]bispropane; 2,3,6,8-tetraoxanonane, 1,1′-[methylenebis(oxy)]bisbutane, 2-methyl-1-[(2-methylpropoxy)methoxy]propane, 2,4,6,8,10-pentaoxaundecane, 2,5,7,10-tetraoxaundecane, 1,3-dioxolane, 1,3-dioxane or 4-methyl-1,3-dioxane; orthoesters, for example 1,1,1-trimethoxymethane, 1,1,1-trimethoxyethane, 1,1,1-trimethoxypropane, 2-methoxy-1,3 -dioxolane, 2-methoxy-2-methyl-1,3-dioxolane, 2-methoxy-2-methyl-1,3-dioxolane, 2-ethoxy-1,3-dioxolane, 2-ethoxy-2-methyl-1,3-dioxolane, 2-ethyl-2-methoxy-1,3-dioxolane, 2-methoxy-1,3-dioxane, 2-methoxy-2-methyl-1,3-dioxane or 2-ethoxy-1,3-dioxane;
carbonates, for example dimethyl carbonate, methyl ethyl carbonate, diethyl carbonate, dipropyl carbonate, diisopropyl carbonate, dibutyl carbonate, dipentyl carbonate, dihexyl carbonate, diheptyl carbonate, dioctyl carbonate, dinonyl carbonate, didecyl carbonate, ethylene carbonate, 4-methyl-1,3-dioxolan-2-one, 4-(methoxymethyl)-1,3-dioxolan-2-one, glycerol carbonate, butylene carbonate, 4,6-dimethyl-3-dioxan-2-one or dibenzyl carbonate;
phosphates, for example trimethyl phosphate, triethyl phosphate, tripropyl phosphate, tributyl phosphate, triisobutyl phosphate, tripentyl phosphate, trihexyl phosphate, tris(2-ethylhexyl) phosphate, tris(2-butoxyethyl) phosphate or triphenyl phosphate;
sulfoxides, for example dimethyl sulfoxide, diethyl sulfoxide, 1,1-dioxotetrahydrothiophene, 1,1-dioxotetrahydro-3 -methylthiophene or 1,1-dioxotetrahydro-2,4-dimethylthiophene.

In addition, the formulations of the invention may optionally comprise liquid fillers e), for example vegetable or mineral oils or esters of vegetable or mineral oils. Suitable vegetable oils e) are all oils which can typically be used in agrochemicals and can be obtained from plants. Examples include sunflower oil, rapeseed oil, olive oil, castor oil, colza oil, corn oil, cottonseed oil, walnut oil, coconut oil and soya oil.

Possible esters are, for example, ethylhexyl palmitate, ethylhexyl oleate, ethylhexyl myristate, ethylhexyl caprylate, isopropyl myristate, isopropyl palmitate, methyl oleate, methyl palmitate, ethyl oleate. Preference is given to rapeseed oil methyl ester and ethylhexyl palmitate. Possible mineral oils are Exxsol® D100 and white oils.

TABLE 2
Illustrative trade names and CAS numbers of preferred compounds e)

| Trade name | Company | General description | CAS No. |
|---|---|---|---|
| Sunflower oil | | Triglycerides of different | 8001-21-6 |
| | | C14-C18 fatty acids, | |
| | | predominantly unsaturated | |
| Rapeseed oil | | Triglycerides of different | 8002-13-9 |
| | | C14-C18 fatty acids, | |
| | | predominantly unsaturated | |
| Corn oil | | Triglycerides of different | 8001-30-7 |
| | | C14-C18 fatty acids, | |
| | | predominantly unsaturated | |
| Soya oil | | Triglycerides of different | 8001-22-7 |
| | | C14-C18 fatty acids, | |
| | | predominantly unsaturated | |
| Rice oil | | Triglycerides of different | 68553-81-1 |
| | | C14-C18 fatty acids, | |
| | | predominantly unsaturated | |
| Radia ® 7129 | Oleon NV, | Ethylhexyl palmitate | 29806-73-3 |
| | BE | | |
| Crodamol ® | Croda, UK | | |
| OP | | | |
| Radia ® 7331 | Oleon NV, | Ethylhexyl oleate | 26399-02-0 |
| | BE | | |
| Radia ® 7128 | Oleon NV, | C12/C14 ethylhexyl | 29806-75-5 |
| | BE | myristate/laurate | |
| Radia ® 7127 | Oleon NV, | Ethylhexyl laurate | 20292-08-4 |
| | BE | | |
| Radia ® 7126 | Oleon NV, | C8/10 ethylhexyl | 63321-70-0 |
| | BE | caprylate/caprate | |
| Estol ® 1514 | Croda | Isopropyl myristate | 110-27-0 |
| Radia ® 7104 | Oleon NV, | Caprylic and capric | 73398-61- |
| | BE | triglycerides, neutral | 5. 65381- |
| | | vegetable oil | 09-1 |
| Radia ® 7732 | Oleon NV, | Isopropyl palmitate | 142-91-6 |
| | BE | | |
| Crodamol ® | Croda, UK | | |
| IPM | | | |
| Radia ® 7060 | Oleon NV, | Methyl oleate | 112-62-9 |
| | BE | | |
| Radia ® 7120 | Oleon NV, | Methyl palmitate | 112-39-0 |
| | BE | | |
| Crodamol ® | Croda | Ethyl oleate | 111-62-6 |
| EO | | | |
| AGNIQUE | Clariant | rapeseed oil methyl ester | 67762-38- |
| ME ® 18 | BASF | | 3. 85586- |
| RD-F, | | | 25-0 |
| Edenor ® | | | |
| MESU | | | |
| Exxsol ® | Exxon Mobil | Hydrotreating low boilers | 64742-47-8 |
| D100 | | (petroleum) | |
| Solvesso ® | ExxonMobil | Solvent naphtha | 64742-94-5 |
| 200ND | | (petroleum), heavy | |
| | | aromatic, naphthalene- | |
| | | depleted | |
| Kristol ® M14 | Carless | White mineral oil | 8042-47-5 |
| Marcol ® 82 | ExxonMobil | (petroleum), C14-C30 | |
| Ondina ® 917 | Shell | branched and linear | |
| Exxsol ®D130 | ExxonMobil | White mineral oil | 64742-46-7 |
| Banole ® 50 | Total | (petroleum) | |
| Genera ®-12 | Total | White mineral oil | 72623-86-0 |
| | | (petroleum) | |
| Genera ®-9 | Total | White mineral oil | 97862-82-3 |
| | | (petroleum) | |

Carrier Materials f) for WGs.

Fillers and carrier materials c2) in the formulations according to the invention are selected from the group comprising minerals, carbonates, sulfates and phosphates of alkaline earth metals and alkali metals, such as calcium carbonate, polymeric carbohydrates, framework silicates, such as precipitated silicas having low absorption, and natural framework silicates, such as kaolin. Typical representatives of suitable fillers f) are, for example, Agsorb® LVM®-GA (attapulgite), Harborlite® 300 (pearlite), Collys® HV (modified starch), Omya® chalk (calcium carbonate), Kaolin® Tec 1 (kaolin, aluminum hydrosilicate), Steamic® OOS (talc, magnesium silicate). For c2), preference is given here to natural framework silicates and calcium carbonate products such as Omya® chalk (calcium carbonate), Kaolin Tec 1® (kaolin) and Harborlite® 300 (pearlite), particular preference to natural framework silicates such as Kaolin®, Tec® 1 (kaolin, aluminum hydrosilicate) and Harborlite® 300 (pearlite). For WG formulations, very particular preference is given to kaolin and calcium carbonate. Further suitable carrier materials or fillers f) are selected from the group of the highly absorptive carriers having an absorption capacity of at least 200 g of dibutyl phthalate per 100 g of carrier material. Preferred highly absorptive carriers f) are silicas, for example Sipernat® products (synthetic precipitated silica of high absorptivity) and fumed silica (Aerosil® products). Preference is given to precipitated silica. The proportion of the fillers f) in the TCs according to the invention is preferably 0.1% to 10% by weight, particularly preferably 0.3% to 8% by weight and very particularly preferably 1% to 7% by weight.

Thickeners g)

Useful thickeners g) include organic thickeners g1) and inorganic thickeners g2).

Useful organic thickeners g1) include organic natural or biotechnologically modified or organic synthetic thickeners. Typical synthetic thickeners are Rheostrux® (Croda) or the Thixin® or Thixatrol® series (Elementis). These are typically based on acrylates. Typical organic thickeners are based on xanthan or cellulose (for instance hydroxyethyl or carboxymethyl cellulose) or a combination thereof. Further typical representatives are based on lignin (such as lignosulfonates, Borresperse®NA, REAX® 88 or Kraftsperse 25 S). Preference is given to using natural modified thickeners based on xanthan. Typical representatives are, for example, Rhodopol® (Solvay) and Kelzan® (Kelco Corp.), and also Satiaxane® (Cargill).

The proportion of the organic thickeners g1) in the SCs according to the invention is not more than 5% by weight, preferably 0.01% to 1.0% by weight, more preferably 0.01% to 0.6% by weight, even more preferably 0.05% to 0.5% by weight and even more preferably 0.1% to 0.3% by weight.

Suitable inorganic thickeners g2) are, for example, modified natural silicates such as chemically modified bentonites, hectorites, attapulgites, montmorillonites, smectites or other silicate minerals such as Bentone® (Elementis), Attagel® (Engelhard), Agsorb® (Oil-Dri Corporation) or Hectorite® (Akzo Nobel), or the Van Gel series (R.T. Vanderbilt).

The proportion of inorganic thickeners g2) in the formulations according to the invention is 0% to 5% by weight, preferably 0.1% to 3% by weight, more preferably 0.2% to 1.5% by weight, even more preferably 0.3% to 1.5% by weight and even more preferably 0.4% to 1.3% by weight.

Preference is given to using a mixture of thickener gl1) and g2). In the case of Scs, particular preference is given to using exclusively organic thickeners d1). Very particular preference is given to those thickeners gl) based on xanthan (such as Rhodopol® G from Solvay).

Further Components h)

In addition, the SCs or TCs or WGs according to the invention may optionally also comprise, as further components h):

wetting agents, pH adjusters, defoamers, biocides, disintegrants, adhesion promoters, antifreezes, preservatives, dyes or fertilizers, and surfactants other than component c).

Suitable defoamers are surface-active silicone- or silane-based compounds such as the Tegopren® products (Goldschmidt), the SE® products (Wacker), and the Bevaloid® (Kemira), Rhodorsil® (Solvay) and Silcolapse® products (Blustar Silicones), preference being given to SE® (Wacker), Rhodorsil® and Silcolapse® products, particular preference, for example, to products such as Silcolapse® 5020.

Suitable antifreezes are those from the group of the ureas, diols and polyols, such as ethylene glycol and propylene glycol, glycerol, preferably propylene glycol or glycerol.

Suitable preservatives are, for example, products such as Acticide® MBS (Biozid, Thor Chemie), CIT, MIT or BIT, for instance Proxel® GXL (BIT), Acticide® SPX (MIT, CIT).

Suitable adhesion promoters may be selected from the group of polyvinylpyrrolidone (PVP), polyvinyl alcohol, copolymer of PVP and dimethylaminoethyl methacrylate, butylated PVP, copolymer of vinyl chloride and vinyl acetate, sodium salt of the copolymer of propenesultanic acid and partially hydrolyzed vinyl acetate, sodium caseinate, phenol resins, modified cellulose types, for example Luviskol® (polyvinylpyrrolidone), Mowiol® (polyvinyl alcohol), modified cellulose.

Suitable antifoams may be selected from the group of the esters of phosphoric acid with lower alcohols, C₆-C₁₀ alcohols, silicone surfactants (suspoemulsions of hydrophobized silica particles in aqueous emulsion concentrates based on liquid silicone surfactants), such as polydimethylsiloxane, and the absorbates thereof onto solid carrier material, for example Rhodorsil® 432 (silicone surfactant), butyl phosphate, isobutyl phosphate, n-octanol, Wacker ASP15 (polydimethylsiloxane, absorbed on solid carrier), Antifoam®SE (polydimethylsiloxane). Preference is given to suspoemulsions of hydrophobized silica particles in aqueous emulsion concentrates based on liquid silicone surfactants, such as Antifoam® SE (polydimethylsiloxane), and solid antifoams, such as Wacker ASP 15 (polydimethylsiloxane).

Further additives h) which may be present in the formulations of the invention are penetrants, wetting agents, spreading agents and/or retention agents. Suitable substances are all of those which can typically be used for this purpose in agrochemicals.

Suitable additives h) are, for example,
ethoxylated branched alcohols (e.g. Genapol® X type) with 2-20 EO units;
ethoxylated branched alcohols with terminal methyl (e.g. Genapol® XM type) with 2-2 EO units;
ethoxylated coconut alcohols (e.g. Genapol® C types) with 2-20 EO units;
ethoxylated C12/15 alcohols (e.g. Synperonic® A types) with 2-20 EO units;
propoxy-ethoxylated alcohols, branched or linear, e.g. Antarox® B/848, Atlas® G5000, Lucramul® HOT 5902;
propoxy-ethoxylated fatty acids with terminal methyl, e.g. Leofat® OC0503M;
organomodified polysiloxanes, e.g. BreakThru® OE444, BreakThru® S240, Silwett® L77,
Silwett® 408;
mono- and diesters of sulfosuccinate sodium salts with branched or linear alcohols having 1-10 carbon atoms;
ethoxylated diacetylenediols (e.g. Surfynol®).

TABLE 3
Illustrative trade names and CAS numbers of preferred compounds h)

| Trade name | Company | General description | CAS No. |
|---|---|---|---|
| Lucramul ® HOT | Levaco | Alcohol ethoxylate-propoxylate | 64366-70-7 |
| 5902 | | (C8-PO8/EO6) | |
| Genapol ® X060 | Clariant | Alcohol ethoxylate (iso-C13- | 9043-30-5 |
| | | EO6) | |
| Genapol ® XM | Clariant | Alcohol ethoxylate (iso-C13- | 345642-79-7 |
| 060 | | EO6/with Me end group) | |
| Triton ® GR 7 ME | Dow | Dioctylsulfosuccinate, sodium | 577-11-7 |
| | | salt | |
| BreakThru ® OE | Evonik | Siloxanes and silicones, cetyl- | 191044-49-2 |
| 444 | Industries | Me, di-Me | |
| BreakThru ® S240 | Evonik | Polyether-modified trisiloxane | 134180-76-0 |
| | Industries | | |
| Silwett ® L77 | Momentive | Polyalkylene oxide-modified | 67674-67-3 |
| | | heptamethyltrisiloxane | |
| Silwett ® 408 | Momentive | Polyalkylene oxide-modified | 67674-67-3 |
| | | heptamethyltrisiloxane | |
| Antarox ® B/848 | Solvay | Oxirane, methyl-, polymer with | 9038-95-3 |
| | | oxirane, monobutyl ether | |
| Atlas ® G5000 | Croda | Oxirane, methyl-, polymer with | 9038-95-3 |
| | | oxirane, monobutyl ether | |
| Leofat ® OC- | Lion | Oxirane, methyl-, polymer with | 181141-31-1 |
| 0503M | Chemical, JP | oxirane, mono-(9Z)-9- | |
| | | octadecenoate, methyl ether, | |
| | | block | |
| Surfynol ® 440 | Air Products | 2,4,7,9-tetramethyldec-5-yne- | 9014-85-1 |
| | | 4,7-diol, ethoxylated | |

Suitable defoamers h are all substances which can typically be used for this purpose in agrochemicals. Preference is given to silicone oils, silicone oil formulations, magnesium stearate, phosphinic acids and phosphonic acids. Examples are Silcolapse® 482 from Bluestar Silicones, Silfoam® SC1132 from Wacker [dimethylsiloxanes and -silicones, CAS No. 63148-62-9], SAG 1538 or SAG 1572 from Momentive [dimethylsiloxanes and -silicones, CAS-No. 63148-62-9] or Fluowet® PL 80.

Possible preservatives h are all substances which can typically be used for this purpose in agrochemicals. Suitable preservatives are, for example, formulations comprising 5-chloro-2-methyl-4-isothiazolin-3-one [CIT; CAS No. 26172-55-4], 2-methyl-4-isothiazolin-3-one [MIT, CAS No. 2682-20-4] or 1,2-benzisothiazol-3(2H)-one [BIT, CAS No. 2634-33-5]. Examples include Preventol® D7 (Lanxess), Kathon® CG/ICP (Rohm & Haas), Acticide® SPX (Thor GmbH) and Proxel® GXL (Arch Chemicals).

Suitable antioxidants h are all substances which can typically be used for this purpose in agrochemicals. Preference is given to butylhydroxytoluene [3,5-di-tert-butyl-4-hydroxytoluene, CAS No. 128-37-0] and citric acid.

Possible colorants h are all substances which can typically be used for this purpose in agrochemicals. Examples include titanium dioxide, carbon black, zinc oxide, blue pigments, red pigments and Permanent Red FGR.

Suitable inert fillers h) are all substances which can typically be used for this purpose in agrochemicals and which do not function as thickeners. Preference is given to inorganic particles such as carbonates, silicates and oxides, and also organic substances such as urea-formaldehyde condensates. Examples include kaolin, rutile, silicon dioxide (“finely divided silica”), silica gel and natural and synthetic silicates, and additionally talc.

The present invention further provides water-dispersible technical concentrates (TCs) based on claim 1, comprising
one or more anionic emulsifiers c2,
one or more nonionic emulsifiers c1),
at least one or more than one carrier material f.
The present invention likewise provides suspension concentrates (SCs) comprising
one or more anionic emulsifiers c2,
one or more nonionic emulsifiers c1,
at least one or more than one thickener g).
The present invention likewise provides ECs comprising
one or more nonionic emulsifiers c1,
at least one solvent e)

The present invention likewise provides ECs comprising
one or more nonionic emulsifiers c1,
at least one further active agrochemical ingredient, preferably a fungicide.

The present invention likewise provides ODs comprising
one or more nonionic emulsifiers c1,
one or more anionic emulsifiers c2,
at least one solvent e)

at least one thickener g)

Application

Examples of administration forms include all the processes known as commonly used to the person skilled in the art: spraying, dipping, misting and a number of specific processes for direct treatment below or above ground of whole plants or parts (seed, root, stolons, stem, trunk, leaf), for example trunk injection in the case of trees or stem bandages in the case of perennial plants, and a number of specific indirect application processes.

The respective area- and/or object-based application rate of the crop protection compositions of a wide variety of different formulation types for control of the harmful organisms mentioned here varies very greatly. In general, the application media known to the person skilled in the art to be commonly used for the respective field of use are used in the conventional amounts for this purpose, for example from several hundred liters of water per hectare in the case of standard spraying processes through a few liters of oil per hectare in the case of ‘ultra low volume’ aircraft application down to a few milliliters of a physiological solution in the case of injection processes. The concentrations of the inventive crop protection compositions in the particular application media therefore vary within a wide range and are dependent on the respective field of use. In general, concentrations known to the person skilled in the art to be commonly used for the respective field of use are used. Preferred concentrations are from 0.01% by weight to 99% by weight, more preferably from 0.1% by weight to 90% by weight.

The agrochemical formulations of the invention can be deployed, for example, in the formulation forms customary for liquid preparations, either as such or after prior dilution with water, i.e., for example, as emulsions, suspensions or solutions. Application is effected by customary methods, i.e., for example by spraying, pouring or injecting.

Depending on the nature of the active ingredient possibly present in addition to prothioconazole, the formulations of the invention are useful for controlling a large number of pests and can be used either for treatment of plant crops or else for that of inanimate material and in the household.

“Pests” or “harmful organisms” are understood here to mean all kinds of pests which can be controlled or kept under control with organic crop protection active ingredients, i.e. crop protection agents, especially fungicides and mixtures of fungicides with other crop protection agents. The term “pest” therefore encompasses organisms that are harmful to plants, especially harmful fungi and their spores, but also harmful insects, arachnids, nematodes and harmful plants. The term “control” encompasses both curative treatment, i.e. the treatment of affected plants with a formulation of the invention, and protective treatment, i.e. the treatment of plants for protection from pest infestation.

The present invention thus also relates to the use of the formulations described herein for control of pests, especially plant pests, and to a method of controlling harmful organisms, especially plant-damaging organisms, comprising the contacting of the harmful organisms, their habitat, their hosts, such as plants and seed, and the soil, the area and the environment in which they grow or could grow, but also of materials, plants, seeds, soil, surfaces or spaces which are to be protected from attack or infestation by organisms that are harmful to plants, with an effective amount of the formulations of the invention.

A further aspect of the invention relates to the use of the formulations described herein for protection of plants including seed, especially useful plants, from infestation by harmful organisms, especially harmful fungi. The present invention thus also relates to the use of the formulations for control of plant-damaging organisms, for example harmful fungi, insects, arachnids, nematodes and harmful plants, especially for control of harmful fungi.

The formulations of the invention can be used in crop protection, particularly as foliar, seed-dressing and soil fungicides, in a manner known per se for control of phytopathogenic fungi.

Plants which can be treated with the formulations of the invention include the following: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Gramineae sp. (for example corn, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soybean), Solanaceae sp. (for example potatoes), Chenopodiaceae sp. (for example sugar beet, fodder beet, swiss chard, beetroot); sugar cane, poppy, olive, coconut, cacao, tobacco and useful plants and ornamental plants in garden and forest; and genetically modified varieties of each of these plants, and also the seeds of these plants.

Preference is given to using the formulations of the invention for treatment of wheat, barley, rye, soya, onions, corn and peanuts.

More particularly, it is possible in principle to use the formulations of prothioconazole of the invention to control all harmful fungi diseases which can also be controlled with the known formulations of prothioconazole. Depending on the particular mixing partner present in each case, the plant diseases are, for example, the following plant diseases:

Alternaria species on vegetables, oilseed rape, sugar beet, soybean, cereals, cotton, fruit and rice (e.g. A. solanior A. alternata on potato and other plants), Aphanomyces species on sugar beet and vegetables, Ascochyta sp. on cotton and rice, Bipo/aris and Drechslera species on corn, cereals, rice and lawn (e.g. teres on barley, D. tritci-repentis on wheat), Blumeria graminis (powdery mildew) on cereals, Botrytis cinerea (gray mold) on strawberries, vegetables, flowers and grapevines, Botryodiplodia sp. on cotton, Bremia lactucae on lettuce, Cerospora species on corn, soybeans, rice and sugar beet (e.g. C. beficula on sugar beet), Cochhobolus species on corn, cereals, rice (e.g. Cochhobolus safivus on cereals, Cochhobolus miyabeanus on rice), Corynespora sp. on soybeans, cotton and other plants, Colletotrichum species on soybeans, cotton and other plants (e.g. C. acutatum on various plants), Curvularia sp. on cereals and rice, Diplodia sp. on cereals and rice, Exserohdum species on corn, Erysiphe cichoracearum and Sphaerotheca fuliginea on cucumber species, Fusarium and Verticillium species (e.g. V. dahliae) on various plants (e.g. F. graminearum on wheat), Gaeumanomyces graminis on cereals, Gibberella species on cereals and rice (e.g. Gibberella fujikuroi on rice), Grainstaining complex on rice, Helminthosporium species (e.g. H. graminicola) on corn and rice, Macrophomina sp. on soybean and cotton, Michrodochium sp. (e.g. M nivale on cereals), Mycosphaerella species on cereals, bananas and peanuts (M graminicola on wheat, M fifiesis on banana), Phaeoisaripsis sp. on soybeans, Phakopsara sp. (e.g. P. pachyrhizi and P. meibomiae on soybeans), Phoma sp. on soybeans, Phomopsis species on soybeans, sunflowers and grapevines (P. viticola on grapevines, P. helianthii on sunflowers), Phytophthora infestans on potatoes and tomatoes, Plasmopara vificola on grapevines, Penecilium sp. on soybeans and cotton, Podosphaera leucotricha on apples, Pseudocercosporella herpotrichoides on cereals, Pseudoperonospora species on hops and cucumber species (e.g. P. cubenis on cucumber), Puccinia species on cerelas, corn and asparagus (P. trificina and P. striformis on wheat, P. asparagi on asparagus), Pyrenophora species on cereals, Pyricularia oryzae, Corficium sasakii, Sarocladium oryzae, S. attenuatum, Entyloma oryzae on rice, Pyricularia grisea on lawn and cereals, Pythium spp. on lawn, rice, corn, cotton, oilseed rape, sunflowers, sugar beet, vegetables and other plants, Rhizoctonia species (e.g. R. solani) on cotton, rice, potatoes, lawn, corn, oilseed rape, potatoes, sugar beet, vegetables and other plants, Rynchosporium sp. (e.g. R. secalis) on rice and cereals, Sclerotinia species (e.g. S. sclerofiorum) on oilseed rape, sunflowers and other plants, Septoria trifici and Stagonospora nodorum on wheat, Erysiphe (syn. Uncinula necator) on grapevines, Setosphaeria species on corn and lawn, Sphacelotheca reilinia on corn, Thievaliopsis species on soybeans and cotton, Tfilefia species on cereals, Ustilago species on cereals, corn and sugar beet, and Venturia species (scab) on apple and pear (e.g. V. inaequalis on apple).

The formulations of the invention can be applied in undiluted form or diluted with water. In general, they are diluted with at least one part water, preferably with 10 parts water and more preferably with at least 100 parts water, for example with 1 to 10 000, preferably 10 to 5000 and more preferably with 50 to 24 000 parts water, based on one part of the formulation.

The present invention likewise provides an emulsion obtainable by mixing water with the liquid formulation of the invention. The mixing ratio of water to emulsion concentrate may be in the range from 1000:1 to 1:1, preferably 400:1 to 10:1.

The dilution is achieved by pouring the emulsion concentrates of the invention into the water. For rapid mixing of the concentrate with water, it is customary to use agitation, for example stirring. However, agitation is generally unnecessary. Even though the temperature for the dilution operation is an uncritical factor, dilutions are typically conducted at temperatures in the range from 0° C. to 50° C., in particular at 10° C. to 30° C. or at ambient temperature.

The water used for dilution is generally tap water. The water may, however, already contain water-soluble or finely dispersed compounds which are used in crop protection, for instance nutrients, fertilizers or pesticides.

It is possible to add various kinds of oils, wetting agents, adjuvants, fertilizers or micronutrients and further pesticides (e.g. herbicides, insecticides, fungicides, growth regulators, safeners) to the emulsion of the invention in the form of a premix or, if appropriate, not until shortly before use (tank-mix). These compositions may be added to the formulations of the invention in a weight ratio of 1:100 to 100:1, preferably 1:10 to 10:1.

The user will apply the formulation of the invention typically from a pre-dosing system, a backpack sprayer, a spraying tank, a spraying aircraft or an irrigation system; the formulation of the invention is typically diluted to the desired deployment concentration with water, buffer and/or further auxiliaries, which affords the ready-to-use spray liquid or agrochemical composition of the invention. Typically, 20 to 2000 liters, preferably 50 to 400 liters, of the ready-to-use spray liquor are deployed per hectare of useful agricultural area.

The required application rates of the pure active ingredients without formulation aids depend on the intensity of pest infestation, on the development phase of the plants, on the climatic conditions of the site of use and on the application method. In general, the application rate is in the range from 0.001 to 3 kg, preferably from 0.005 to 2 kg, more preferably from 0.01 to 1 kg and most preferably from 50 to 500 g of active ingredient per hectare, active ingredient here meaning prothioconazole plus possible further active ingredients.

The generally diluted formulations of the invention are applied mainly by spraying, especially spraying of the leaves. Application can be conducted by spraying techniques known to those skilled in the art, for example using water as carrier and amounts of spray liquor of about 50 to 1000 liters per hectare, for example from 100 to 400 liters per hectare.

The novel prothioconazole-containing formulations have advantageous properties in respect of the treatment of plants; more particularly, they feature good use properties, high stability and high fungicidal activity.

The invention is illustrated in detail by the examples but is not restricted thereto.

### EXAMPLES

### Feedstocks Used

The terms used in the examples below have the following meanings:

| | |
|---|---|
| prothioconazole | PTZ (Bayer AG) |
| Synperonic F127 | propylene oxide/ethylene oxide (PO-EO) |
| | block polymer (Croda) |
| Soprophor 4D384 | tristyrylphenol ethoxylate sulfate ammonium |
| | salt (Solvay) |
| citric acid | polybasic organic acid |
| Rhodopol ® 23 | xanthan derivative (Solvay) |
| Silcolapse ® 411 | silicone defoamer (Solvay) |
| Silfoam ® SE 2 | silicone defoamer (Wacker) |
| glycerol | antifreeze |
| Proxel ® GXL | preservative (biocide, Proxel) |
| Kaolin TEC1 | aluminum hydrosilicate |
| Sipernat ® 22S | precipitated silica |
| Sokalan ® K30 | polyvinylpyrrolidone, nonionic dispersant |
| Solvesso 200 ND | mineral oil, ExxonMobil, naphthalene-free |
| Calsogen ® 4814 | calcium dodecylbenzenesulfonate in |
| | 2-ethylhexanol (about 40%), Clariant |
| Emulsogen ® 3510 | nonionic surfactant, n-butyl-PO-EO block |
| | oxalkylate, Clariant |
| Lucramul ® CO30 | nonionic emulsifier, castor oil ethoxylated, |
| | Levaco Chemicals, DE |
| Rhodocal 60BE | calcium dodecylbenzenesulfonate in |
| | 2-ethylhexanol (about 40%), Solvay |
| EL400 | ethoxylated (40EO) castor oil, Croda |
| Aerosil 972 | hydrophobic finely divided silicic acid, Evonik |
| Soprophor ® 796/P | nonionic emulsifier, ethoxylated |
| | propoxylated tristyrylphenol, Solvay |
| SAG1572 | aqueous emulsion of polydimethylsiloxane |
| | Momentive ® |
| Hallcomid ® 1025 | dimethyldecenamide Stepan |
| Genagen ® 4296 | dimethyldecanamide Clariant |
| Agnique ® KE 3308 | dimethyldecanamide BASF |
| 9-decenoic acid | Sigma-Aldrich (CAS number: 14436-32-9) |
| limonene | Sigma-Aldrich, available as various isomers under |
| | the following CAS numbers 5989-27-5 [(D)-(+)- |
| | limonene], 5989-54-8 [(S)-(−)-limonene], |
| | 138-86-3 [dipentene unspecified], 7705-14-8 [(±)- |
| | limonene], 6876-12-6 [trans-1-methyl-4- |
| | (methylvinyl)cyclohexene |

General Preparation of an Aqueous Suspension Concentrate (SC):

First of all, water is initially charged at room temperature. The active ingredients and the other components are added (in no particular order) with stirring. The mixture is pre-comminuted in a colloid mill, followed by wet grinding using, for example, a bead mill. Finally, the organic thickener is added.

General Preparation of Water-Dispersible Granules (WG)

Water is initially charged at room temperature. The active ingredients and the other components are added (in no particular order) with stirring. The mixture is pre-comminuted in a colloid mill, followed by wet grinding using, for example, a bead mill. The TC slurries according to the invention obtained in this manner are processed further to give the WG. Fluidized bed drying is carried out according to the prior art.

General Preparation of an Emulsion oncentrate (EC)

The organic solvent is initially charged. All other components are then added (in no particular order) with stirring. Stirring is continued until a clear solution is formed.

General Preparation of an Organic Dispersion Concentrate (OD):

First, the organic solvent is initially charged at room temperature. The active ingredients and the other components are added (in no particular order) with stirring. The mixture is pre-comminuted in a colloid mill, followed by wet grinding using, for example, a bead mill.

Determination of 2-(1-chlorocyclopropyl)-1-(2-chlorophenyl)-3-(1H-1,2,4-triazol-1-yl)propan-2-ol (Compound III) in Formulations

2-(1-Chlorocyclopropyl)-1-(2-chlorophenyl)-3-(1H-1,2,4-triazol-1-yl)propan-2-ol is separated from the formulation constituents on a reverse phase column using an isocratic eluent. After MS/MS detection, the quantitative evaluation is conducted by comparing the peak areas with those of the reference object, using an external standard.

High-pressure liquid chromatograph: HP 1090

Sample injection: HP 1090 Autoinjector

Mass spectrometer: Quattro I, Fisons

Integration and evaluation: MassLynx from Micromass

In each case, the samples are, unless indicated otherwise, exposed to light and stored for 4 weeks.

Example 1: Recipe of a Prothioconazole Emulsion Concentrate Formulation (Storage in the Dark)

| | 1a | |
|---|---|---|
| | % by | |
| Component | weight | 1b |
| prothioconazole | 12.75 | 13.00 |
| bixafen | 6.37 | 6.5 |
| fluopyram | 6.37 | 6.5 |
| Lucramul HOT 5902 | 7.50 | 7.50 |
| Lucramul CO 30 | 7.50 | 7.50 |
| Soprophor 796/P | 5.00 | 5.00 |
| SAG1572 | 0.10 | 0.10 |
| Genagen ® 4296 (comparison) | 54.41 | 43.90 |
| 9-decenoic acid (according to the invention) | | 10.00 |

| | Compound |
|---|---|
| | (III) |
| | content % |
| | by weight |
| Example 1a | 0.0037 |
| (comparison) | |
| Example 1b | 0.0023 |

Example 2: Recipe of a Prothioconazole Suspension Concentrate

| | 2a | |
|---|---|---|
| | % by | |
| Component | weight | 2b |
| prothioconazole | 45 | 45 |
| Pluronic PE 10500 | 6.0 | 6.0 |
| urea | 10 | 10 |
| defoamer (Momentive ® SAG1572) | 0.1 | 0.1 |
| Proxel GXL | 0.2 | 0.2 |
| Rhodopol G | 0.2 | 0.2 |
| water | 28.5 | 28.5 |
| water (comparison) | 10 | |
| 9-decenoic acid | | 10 |

| | Compound |
|---|---|
| | (III) |
| | content % |
| | by weight |
| Example 2a | 0.0026 |
| (comparison) | |
| Example 2b | 0.0024 |

Example 3: Recipe of a Prothioconazole WG (Waterdispersible Granules, Fluidized Bed Granules)

| | 3a | | |
|---|---|---|---|
| | % by | | |
| Component | weight | 3b | 3c |
| prothioconazole | 50 | 50 | 50 |
| Baykanol SL | 15 | 15 | 15 |
| Oparyl MT 804 | 1 | 1 | 1 |
| Kaolin W | 29 | 29 | 29 |
| Kaolin W (comparison) | 5 | 5 | 5 |
| 9-decenoic acid | | 5 | |
| 9-decenoate ester with 10 EO groups | | | 5 |

| | Compound |
|---|---|
| | (III) |
| | content % |
| | by weight |
| Example 3a | 0.0094 |
| (comparison) | |
| Example 3b | 0.0071 |
| Example 3c | 0.0077 |

Example 4: Recipe of a Prothioconazole WG (Waterdispersible Granules, Prepared as Extrusion Granules)

| | 3a | | |
|---|---|---|---|
| | % by | | |
| Component | weight | 3b | 3c |
| prothioconazole | 50 | 50 | 50 |
| Baykanol SL | 20 | 20 | 20 |
| Pergopak M | 5 | 5 | 5 |
| NaHCO3 | 20 | 20 | 20 |
| NaHCO3 (comparison) | 5 | 5 | 5 |
| 9-decenoic acid | | 5 | |
| 9-decenoate ester with 10 EO groups | | | 5 |

1. A formulation, comprising
a) prothioconazole and
b) at least one compound of formula (IV) 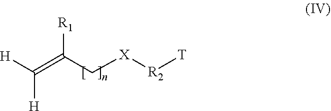
where
n=0-24; optionally 2-16; optionally 4-14; and optionally 8-12:
R₁═H, C₁-C₆-alkyl, —CN, Cl, Br, F, optionally H, C₁-C₄-alkyl, ptionally H, methyl; and optionally H;
X═—COO—, —CONR₃R₄, —S—, —SO₂—, —O— and —COS—; preferably —COO—, CONR₃R₄, —S—, —O— and —COS—; optionally —COO—, CONR₃R₄; and optionally —COO—;
R₂═(—CH₂—)ₘ, (—CH₂O—)ₘ, (—C₂H₄O—)ₘ, (—C₄H₆O—)ₘ, optionally (—CH₂—)ₘ, (—CH₂O—)ₘ, (—C₂H₄O—)ₘ, (—C₃H₆O—)ₘ; optionally (—CH₂O—)ₘ, (—C₂H₄O—)ₘ, (—C₃H₆O—)ₘ; and optionally (—CH₂O—)ₘ,
T=H, sulfate, phosphate, C₁-C₃-alkyl, —OH, —SH, sulfone; ptionally H, sulfate, sulfone, —OH, phosphate; optionally H, sulfate, —OH; and particularly optionally H and OH;
R₃═H, C₁-C₆-alkyl, C₃-C₆-cycloalkyl, C₆-C₁₀-aryl, ptionally H, C₁-C₄-alkyl, C₅-C₆-cycloalkyl, phenyl, optionally H, methyl and phenyl; and optionally H;
R₄═H, methyl, ethyl, propyl, (—CH₂—)ₘ, (—CH₂O—)ₘ, (—C₂H₄O—)ₘ, (—C₃H₆O—)ₘ, (—C₄H₈O—)ₘ; optionally H, methyl, ethyl, propyl, (—CH₂—)ₘ, (—CH₂O—)ₘ, (—C₂H₄O—)m, (—C₃H₆O—)ₘ; more optionally H, methyl, ethyl, propyl, (—CH₂O—)ₘ, (—C₂H₄O—)ₘ, (—C₃H₆O—)ₘ; and particularly optionally H, methyl, ethyl, propyl, (—CH₂O—)ₘ,
and R₃ is different from R₄, and
m=0-100, optionally 2-50, optionally 5-25, and optionally 8-12;
where the formulation is free from compounds according to formula (I) 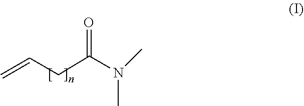
in which
n is 3, 4, 5, 6, 7, 8, 9, 10, 11, 12, 15 or 18,
and additionally free from glycoside surfactants having a 9-decenoyl radical,
where
for X═—CNR₃R₄ and m=0 T is not present.

2. The formulation as claimed in claim 1, wherein in formula (IV)
n=2-16;
R₁═H, C₁-C₄-alkyl,
X═—COO—, CONR₃R₄, —S—, —O— and —COS—; R₂═(—CH₂—)ₘ, (—CH₂O—)ₘ, (—C₂H₄O—)ₘ, (—C₃H₆O—)ₘ;
T=H, sulfate, sulfone, —OH, phosphate;
R₃═H, C₁-C₄-alkyl, C₅-C₆-cycloalkyl, phenyl;
R₄═H, methyl, ethyl, propyl, (—CH₂—)ₘ, (—CH₂O—)ₘ, (—C₂H₄O—)ₘ, (—C₃H₆O—)ₘ;
and R₃ is different from R₄, and
m=0-50.

3. The formulation as claimed in claim 1, wherein in formula (IV)
n=8-12:
R₁═H;
X═—COO—,
R₂═(—CH₂O—)ₘ,
T=H and OH;
m=8-12.

4. The formulation as claimed in claim 1, wherein in formula (IV)
n=7:
R₁═H;
X═—COO—;
T=H;
m=0.

5. The formulation as claimed in claim 1, wherein the compound IV is selected from the group consisting of
9-decenoic acid, 9-decenoate ester having n EO groups (EO═C₂H₄O), where n=8, 9, 10, 11 and optionally n=10;
C10-17 DMAPA-amides 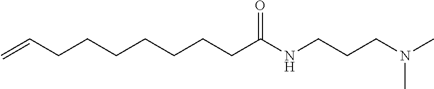 [SMILES: C=CCCCCCCCC(=O)NCCCN(C)C]
C10-20 DMAPA amine-oxides 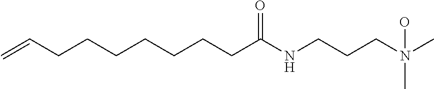
and
C10-24: sulfobetaines.

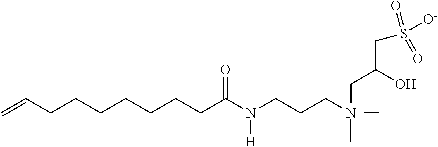 [SMILES: C=CCCCCCCCC(=O)NCCC[N+](C)(C)CC(O)CS(=O)(=O)[O-]]

6. The formulation as claimed in claim 1, wherein the proportion of component b) is 1% by weight to 50% by weight.

7. The formulation as claimed in claim 1, wherein the proportion of component a) is 1% by weight to 50% by weight.

8. The formulation as claimed in claim 1, wherein this formulation comprises as least one of the following components:
c) c1: non-ionic dispersant/emulsifier,
c2: ionic dispersant/emulsifier
d) one or more other active agrochemical ingredients different from a)
e) one or more solvents including OD carriers
f) one or more carriers (WGs, and also Aerosils for SC/TK/WG)
g) one or more of: g1: organic thickeners
g2: inorganic thickeners
h) one or more further additives and auxiliaries.

9. The formulation as claimed in claim 8, comprising a nonionic (c1) and/or anionic (c2) emulsifier.

10. The formulation as claimed in claim 8, wherein said one or more active agrochemical ingredients d) are one or more active insecticidal or fungicidal ingredients.

11. The formulation as claimed in claim 8, wherein the further active agrochemical ingredient d) is bixafen.

12. The formulation as claimed in claim 10, comprising not more than 5% by weight of water.

13. A process for preparing the formulation as claimed in claim 1 comprising mixing components a) and b) and optionally one or more further additives.

14. A method of controlling one or more harmful organisms, comprising contacting the harmful organisms, a habitat, one or more hosts, optionally plants and seed, and/or soil, an area and/or an environment in which said organisms grow or could grow, and/or one or more materials, plants, seeds, soil, surfaces or spaces which are to be protected from attack or infestation by organisms that are harmful to plants, with an effective amount of the formulation as claimed in claim 1.

15. The use of the formulations as claimed in claim 1 for protection of plants including seed, and/or optionally useful plants, from infestation by one or more harmful organisms.

16. The formulations as claimed in claim 1 for control of one or more organisms that are harmful to plants, optionally phytopathogenic harmful fungi, insects, arachnids, nematodes and harmful plants.

17. The formulation as claimed in claim 15, wherein the harmful organisms are phytopathogenic harmful fungi.

18. An emulsion obtainable by mixing water with the formulation as claimed in claim 1, wherein the mixing ratio of water to emulsion concentrate is from 1000:1 to 1:1.
